(19) United States
(12) Patent Application Publication
(10) Pub. No.: US 2004/0102328 A1
(43) Pub. Date: May 27, 2004

(54) Thermo-stable bio-matrix

(76) Inventors: Von Walter Johnson, Christchurch (NZ); Joan Frances Pearson, Christchurch (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/362,465

(22) PCT Filed: Aug. 22, 2001
(86) PCT No.: PCT/NZ01/00167

(30) Foreign Application Priority Data
Aug. 22, 2000 (NZ) 506484
Aug. 22, 2000 (NZ) 506485

(51) Int. Cl. A61K 39/04; A01N 25/28; A61K 9/14

(52) U.S. Cl. 504/359; 424/488; 424/248.1

(57) ABSTRACT

A method for producing a thermo-stable biodegradable medium for storage of biological materials is disclosed. The medium contains a bio polymer selected from the group of xanthan gum, acacia gum, guar gum, gellan, starch or a combination thereof. The biological material can be a wide range of materials including; a bio-inoculant such as Rhizobium, a vaccine, a microorganism, an enzyme, a protein or a pharmaceutical. The medium can be used for medium to long term storage of material at room temperature with a half life of 50% for up to 6 months. The medium is water soluble and can be used as a fertilizer, spray or an inoculant.

### TECHNICAL FIELD

The present invention relates to a process and product for the stabilisation and storage of biological materials and bio-compatible materials. More particularly the present invention relates to a process for producing a bio-polymer matrix for the stabilisation and storage of such materials.

### BACKGROUND ART

For the purpose of this specification the term “biological materials” is used to encompass, but is not limited to, any or all of the following: a bio-inoculant, a micro-organism, biological cells, a part or parts of biological cells, pharmaceuticals, enzymes, hormones, proteins and other bio-chemicals, unstable compounds and compositions (both biological and non-biological); and a combination of these.

A known problem associated with the industrial or agricultural application of biological materials is the maintenance of the materials in a viable state or a stable state until they are used, or during the period of time they may be incorporated in a slow release delivery mechanism. Many biological materials cannot be maintained in a viable condition during storage, particularly where they are not kept or can not be kept under refrigeration. This is a particular problem with non-spore forming bacteria.

At present, use of bacterial products as the biological material requires production of high concentrations of bacteria to ensure survival of commercially useful numbers for extended periods. This has been achieved to a limited degree using refrigeration and/or freeze drying to preserve viability. Additionally, while some microbial products require only the delivery of an inoculative dose, for others (such as bio-pesticides), delivery of a higher minimum dosage concentration is essential to the success of the product.

A number of different formulations and media have been proposed, used and disclosed in order to overcome this “shelf-life” problem. Some formulations emphasise the selection of the basic active ingredient for the storage matrix “the bio-polymer”, whilst others disclose methods for preparation of this matrix, or the method of introduction of the biological agent into the matrix and the conditions under which any of these steps occur.

### CONDITIONS

WO98/13471 discloses a formulation formed from polyvinylpyrrolidone (PVP). With the use of this active ingredient as the matrix, some biological material is found to survive for at least 8½ months when stored in vacuum packaging in a temperature range of 5-25° C.

U.S. Pat. No. 4,434,231 discloses a polymer matrix which is partially cross-linked and comprised of a gel of one or more polymers. The gel is dried and it was found that the biological agent was not converted to a dormant or latent state. The partially cross-linked polymer is effected by one of the following: heat treatment, metallic salt action, introduction of a further polymer or another polysaccharide. Additionally, it discloses the gel as being prepared at elevated temperatures, prior to the introduction of the liquid culture of the biological agent. Further complexity is added in the examples disclosed to show the viability of the one selected organism, Rhizobium japonicum.

WO98/13471 also discloses that vacuum packaging significantly decreases the practicality of the commercial production of the product.

### FORMULATIONS

U.S. Pat. No. 4,155,737 discloses the use of the polymer, polyacrylamide. Use of xanthan, carob, carrageenan, and sodium alginate is disclosed in U.S. Pat. Nos. 4,434,231, 5,021,350, 5,292,507. WO98/13471 discloses PVP as enhancing survival of sensitive micro-organisms.

U.S. Pat. No. 5,292,507 discloses and addresses the necessity for additional steps and preparation of gels to avoid the handling that is attendant on use of such gums as xanthan gum. However, this patent discloses only the use of a liquid system which specifically avoids semi-solids, viscous gels or have gum-like properties. It discloses the use of polysaccharide and polymers that are not cross-linked or are not substantially cross-linked where the degree of cross-linking is less than 10%.

U.S. Pat. No. 5,292,507 discloses a method for suspending bacterial cells in a non crosslinked polysaccharide solution, and incorporation into an oil emulsion. This solution is then diluted with water and used in a liquid spray either for direct application or for coating of seeds. The solution may also be reduced to a powder. Finally, the liquid solution prepared has by weight 0.05% to 10% non cross-linked polysaccharide.

U.S. Pat. No. 5,113,619 discloses a composition which includes bacteria and an adherent which is a bio-polymer. The bio-polymer acts as a matrix for protecting the bacteria, which is applied to a seed.

As can be seen from the preceding patents, many disclose the advantageous use of two bio-polymers. However such use adds to the handling costs, leading to a more expensive production technique than the use of a single bio-polymer.

The above discussed matrices formed in pourable liquids also require that transportation costs are higher than they might otherwise be. Further, processing treatments to the liquid are also higher than are needed.

### OTHER PREPARATION METHODS

Other two-stage processes for deriving a matrix for stable storage of biological agents can be found in U.S. Pat. No. 4,954,443. This discloses the use of a first and a second aqueous solution for the immobilisation of enzymes and micro-organisms. The first solution contains at least one immobilising agent, which can be xanthan gum or its derivatives. The second aqueous solution includes metal ions having a valence of three or more. After the two solutions are combined the immobilising agent is thereby hardened into a state in which it encloses the biological agents.

However, as with previous methods of producing biological storage medium, non-biodegradable or toxic elements are introduced into the process to form the storage medium. Further, this invention does not disclose any survival rates of microorganisms and thus may not be useful for agricultural or environmental applications, especially with respect to bio-inoculants.

It is an object of the present invention to provide a process for producing a storage medium for biological materials which is simple, easy to effect, and produces a non-toxic bio-degradable matrix, without reducing the efficiency of the storage, stabilisation or preservative characteristics of the bio-matrix at room temperature and pressure.

It is a further object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method for producing a thermo-stable bio-degradable medium for storage of biological materials, said method including the steps of:

(a) preparing at least one bio-polymer at a concentration of 100-10% by weight of a mixture at room temperature, said mixture being in a state selected from a solid and a suspension;

(b) preparing a concentrate of the biological materials of between 10% and 100% (by weight), said concentrate being in a state selected from a solid and a suspension;

(c) combining the mixture of step (a) and the preparation (b), to form a second mix; and

(d) agitating the second mix at room temperature to form a homogeneous suspension; wherein a gel is formed;

and wherein the bio-polymer is selected from the group: xanthan gum; acacia gum; guar gum; gellan; starch; and a combination thereof;

and wherein the biological material is selected from the group: a bio-innoculant, a micro-organism, biological cells, part of a biological cell, parts of a biological cell, a vaccine, at least one pharmaceutical compound, at least one enzyme, at least one hormone, at least one protein; at least one bio-chemical, biological unstable composition; at least one non-biological compound; and a combination of these.

According to a further aspect of the present invention there is provided a method for producing a thermo-stable bio-degradable medium for storage of biological materials wherein the biological material includes: a pesticide; a viricide; a bacteriacide; a fungicide; and a combination of these.

According to a further aspect of the present invention there is provided a method for producing a thermo-stable biodegradable medium for storage of biological materials wherein the biological material is a vaccine selected from: a live vaccine; an oral attenuated vaccine; an encapsulated myco bacterium vaccine; and a combination of these. Examples of the vaccine include Bacille Calmette and Guerin (B.C.G.).

According to a further aspect of the present invention there is provided a method of producing a thermo-stable biodegradable medium as described above wherein within step (c) the ratio of the mixtures of steps (a) and (b), which are combined in step (c), is in the range 1:10 to 10:1 by weight. The range is optimally 1:1.

According to a further aspect of the present invention there is provided a method of producing a thermo-stable biodegradable medium as described above wherein said biological material is between 10 to 20% by weight in the concentrate of step (b).

Optimally, the second mix should be allowed to stand at room temperature after step (d) and the time should be approximately 60 minutes since being made.

According to a further aspect of the present invention there is provided a method for producing a thermo-stable bio-degradable medium for storage of biological materials wherein said method includes, a further step, before step (d):

(ci) adding a biodegradable non-toxic oil to the mix, the concentration of oil being in the range 0.1 to 10% by weight of the mix.

Optimally also, the oil is in the range of 1% to 10% by weight of the mix.

Optionally, the oil used in step (d) may be any biodegradable, monounsaturated oil which can be used in a refined or non-refined state. The oil may include a combination of oils, which may or may not be edible, as is desired. For example, olive oil, canola oil, sunflower seed oil, and hydrolysed oils may be used as is desired.

According to a further object of the present invention there is provided a method of producing a thermo-stable biodegradable medium as described above wherein the biological material is cellular or a micro-organism. The concentration of such biological material, at the end of step (d) is hereafter referred to as the “cell concentration”. Advantageously, the cell concentration is in the range 10⁵ cells to 10¹² cells g⁻¹, more preferably in the range 10⁸ to 10¹² cells g⁻¹, more preferably in the range of 10⁹ to 10¹⁰ cell g⁻¹. Advantageously, the biological material may be present in the concentrate of step (b) in a broth, or on a growing medium.

According to another aspect of the present invention there is provided a method of producing a thermo-stable biodegradable medium as described above wherein the biological material introduced is a micro-organism. The micro-organism is selected from the group: Serratia, Pseudomonas, Xanthomonas and Rhizobium, and a combination thereof.

Optionally, the bio-polymer is xanthan gum, or a mixture of xanthan and acacia gums, which is added as a dry solid in a ratio in the range of 1:2 to 1:6 by weight.

It will be appreciated that more than one bio-polymer and/or more than one biological agent may be present in the steps (a) to (c) as described above.

According to a further object of the present invention there is provided a method of producing a thermo-stable biodegradable medium as described above wherein said method includes: the steps of (a) to (c) with at least one first bio-polymer; the steps of (a) to (c) with at least one second bio-polymer; and a mixing of these two mixtures by steps (c) and (d) as described above.

For the purposes of the specification, the term “storage” means a stability of better than LT₅₀ with respect to the cell concentration of the biological material. That is, more than 50% of the cells (if cells are the biological material) are viable at the end of the storage period; or more than 50% of the non-living material is viable at the end of the storage period. Advantageously, LT₅₀ may be achievable after 2 months, 4 to 6 months, or 12 to 18 months.

For the purposes of the specification the term “thermo-stable” means a range of temperatures in which the combination of bio-polymer and biological material is stable. This temperature range is 4° C. to 40° C. preferably between 5° C. to 30° C.

According to a further aspect of the present invention there is provided the method as described above wherein said method includes a further step (e):

(e) spreading the gel to 5-10 mm in thickness and air-drying it to a moisture content in the range 0.05% to 20% by weight.

Optionally, step (e) takes from 12 to 17 hours at ambient or room temperature. Optionally also, the gel is at a thickness of 5 mm before drying. Optionally the moisture content is approximately 20% by weight at the end of the drying.

According to a yet further aspect of the present invention there is provided a thermo-stable biodegradable medium prepared by the above described methods.

According to another aspect of the present invention there is provided a biological storage medium, in the form of a gel of less than 95% by weight of water, produced by the method as described above.

For the purposes of this specification the term “substrate” is used to encompass, but is not limited to, agricultural, horticultural, forestry or other commercial substrates, such as grasses and crops, soils (etc); water, waste water, skins of animals and tissues of animals; and solids such as sands and gravels and other uncultivated and friable materials.

According to a further aspect of the present invention there is provided a liquid spray for application to a substrate, said spray at least including:

a portion of thermo-stable biodegradable medium as described above; and

a liquid carrier.

Preferably, the medium can be added to a trickle irrigation system.

According to a further aspect of the present invention there is provided a liquid spray for application to a substrate, wherein said substrate is selected from: an agricultural crop; a horticulture crop; a forestry crop; the outer layer of an animal; an uncultivated surface; and a combination thereof.

According to a still further aspect of the present invention there is provided a method of inoculating a plant seed with a biological material, said method including the steps of;

(a) selecting at least one biological material to be used as an inoculant;

(b) preparing the medium composition by the above described method;

(c) adding the composition to water and mixing to release the biological material into the solution;

(d) soaking the plant seed in said solution to allow the biological material to coat the plant seed.

According to a further aspect of the present invention there is provided a method of inoculating a plant seed with a biological material substantially as describe above, said method further including, after step (b), the step (bi): adding a powdered compound to the matrix composition, said powdered composition being selected from the group: a second biological material, a dried and powdered granule composition, a dried and powdered bio-polymer matrix containing a second or a third biological material, a chemical, and a combination of these.

Optimally, the plant seed can be dried at room temperature before drilling or seed broadcast. Optimally, more than one inoculant may be used in step (a) above, each being for a different purpose. As the medium is thermo-stable and bio-stable, the seeds need not be drilled or sown immediately after the inoculation process.

According to a further aspect of the present invention there is provided seed, inoculated by the method as described above.

According to a further aspect of the present invention there is provided seed inoculated by a medium composition wherein the seed is drilled in combination with a dried medium composition.

It can be seen from the above described invention that storage of a biological material can be effected without the need for special conditions, and after a simple preparation process for the bio-matrix used as the storage medium.

### BEST MODES FOR CARRYING OUT THE INVENTION

### EXAMPLE 1

For each microorganism test below, 7.5 grams of dried xanthan gum is added to 135 grams of concentrated biological material by agitation at room temperature to form a homogenous mix.

This mix is left for 1 hour at room temperature. 7.5 grams of pure canola oil is added and the suspension is agitated for 10-15 minutes at room temperature.

A gel is made with the bio-polymer xanthan and one of each of a range of microorganisms: Serratia entomophila, Serratia marcescens, Pseudomonas aeruginosa, Rhizobium leguminosarum (biological materials). Each of these micro-organisms is at a cell concentration of approximately 10⁹ to 10¹⁰ cells g¹. The cell concentrations are set out in Table 1.

The survival rate of the micro-organisms is tested and the results are set out in Table 1.

TABLE 1
Example 1

| Sample | | Initial | 1 mth | 2 mth | 3 mth | 4 mth | 6 mth | LT₅₀ |
|---|---|---|---|---|---|---|---|---|
| # | Organism | cfug⁻¹ | cfug⁻¹ | cfug⁻¹ | cfug⁻¹ | cfug⁻¹ | cfug⁻¹ | Days |
| T213 | Serratia | 3.6 × 10¹⁰ | — | — | 1.16 × 10¹⁰ | — | 1.27 × 10¹⁰ | ˜180 |
| | entomophila | (theo) | | | | | | |
| T198 | Serratia | 2.61 × 10¹⁰ | 1.06 × 10¹⁰ | — | 1.78 × 10¹⁰ | — | 1.75 × 10¹⁰ | >180 |
| | entomophila | (theo) | | | | | | |
| PT246 | Serratia | 1.51 × 10¹⁰ | — | 1.27 × 10¹⁰ | — | 1.26 × 10¹⁰ | — | >120 |
| | marcescens | | | | | | | |
| PT280 | Pseudoinonas | 3.25 × 10¹⁰ | — | 3.19 × 10¹⁰ | — | — | — | >60 |
| | aeruginosa | | | | | | | |
| PT284 | Rhizobium | 1.39 × 10⁹ | — | 1.89 × 10⁹ | — | — | — | >60 |
| | leguminosarum | | | | | | | |

Comparison

| | Initial | 1 week | 2 week | 3 week | 4 week | LT₅₀ |
|---|---|---|---|---|---|---|
| Organism | cfug⁻¹ | cfug⁻¹ | cfug⁻¹ | cfug⁻¹ | cfug⁻¹ | Days |
| Serratia | 6.67 × 10¹⁰ | 4.52 × 10¹⁰ | 1.22 × 10⁸ | 3.83 × 10⁸ | 9.97 × 10⁷ | <14 |
| entomophila | | | | | | |
| Serratia | 8.93 × 10¹⁰ | 7.84 × 10¹⁰ | 6.16 × 10¹⁰ | 4.36 × 10¹⁰ | 1.58 × 10¹⁰ | <28 |
| marcescens | | | | | | |
| Pseudomonas | 7.34 × 10¹⁰ | 1.01 × 10¹⁰ | 6.93 × 10⁸ | 2.51 × 10⁸ | 1.51 × 10⁸ | <7 |
| aeruginosa | | | | | | |

A further example is also included, T213, using the above method wherein 5 grams of xanthan gum, 5 grams of oil and 90 grams of concentrated biological material containing Serratia entomophila are used.

By comparison, tests were done showing the rate of microbe survival for the microbe in a broth at 20° C. The results are shown above in Table 1 under the heading comparison.

### EXAMPLE 2

Separate gels are made with the bio-polymer starch and one each of a range of microorganisms: Serratia marcescens, Pseudomonas aeruginosa, Rhizobium leguminosarum (biological materials). Each of these micro-organisms is concentrated at approximately 10¹⁰ cells g⁻¹. The cell concentrations are set out in Table 2.

For each composition 15 grams of starch is added to 100 grams of microbial concentrate. The mix is agitated for 10 minutes at room temperature. The resultant gel matrix is stored in a plastic bag at a shelf temperature of approximately 20° C. for up to 2 months.

The survival rate of the micro-organisms were tested and the results are shown in the attached Table 2.

TABLE 2
Example 2

| | | Initial | 2 month | LT₅₀ |
|---|---|---|---|---|
| Sample | Organism | cfu g⁻¹ | cfu g⁻¹ | Days |
| PT248 | Serratia | 1.89 × 10¹⁰ | 3.45 × 10¹⁰ | >60 |
| | marcescens | | | |
| PT282 | Pseudomonas | 2.89 × 10¹⁰ | 1.27 × 10¹⁰ | ˜60 |
| | aeruginosa | | | |
| PT286 | Rhizobium | 6.26 × 10⁸ | 1.28 × 10⁹ | >60 |
| | leguminosarum | | | |

### EXAMPLE 3

Separate gels are made with the bio-polymer xanthan and one of each of a range of micro-organisms: Serratia entomophila, Serratia marcescens, Pseudomonas aeruginosa (biological materials). Each of these micro-organisms is concentrated at approximately 10¹⁰ cells g⁻¹. The cell concentrations are set out in Table 3.

For each sample, to 7.5 grams dry xanthan gum is added 42.5 grams distilled water. The mixture is agitated at room temperature for between 5-10 minutes to form a suspension. Alternatively a 50% solution of xanthan gum medium may be used.

50 grams of each micro-organism concentrate is added to the respective suspension. The mix is agitated for a further 10 minutes at room temperature. The resultant gel matrix is stored in a plastic bag at a shelf temperature of approximately 20° C. for up to 2 months.

The survival rate of the micro-organisms are tested and the results are set out in the attached Table 3.

TABLE 3
Example 3

| | Initial | Survival - 2 months |
|---|---|---|
| Organism | Concentration cfu g⁻¹ | cfu g⁻¹ |
| Serratia entomophila | 3.32 × 10¹⁰ | 1.51 × 10¹⁰ |
| Serratia marcescens | 4.47 × 10¹⁰ | 1.73 × 10¹⁰ |
| Pseudomonas aeruginosa | 1.05 × 10¹⁰ | 7.63 × 10¹⁰ |

### EXAMPLE 4

The gels from Example 1 are each spread out to a 5 mm thickness and air dried at room temperature for 15-20 hours. The dry gels are each stored in a plastic container at room temperature for up to 6 months.

The survival rate of the micro-organisms are tested and the results are set out in the attached Table 4.

TABLE 4
Example 4

| | Initial | Survival | Survival |
|---|---|---|---|
| | Concentration | 1 month | 2 months |
| Organism | cfu g⁻¹ | cfu g⁻¹ | cfu g⁻¹ |
| Serratia entomophila | 7.03 × 10¹⁰ | 7.99 × 10⁸ | — |
| Serratia marcescens | 1.27 × 10¹⁰ | 8.36 × 10⁸ | 3.62 × 10⁷ |
| Pseudomonas aeruginosa | 8.30 × 10¹⁰ | 3.26 × 10¹⁰ | 4.95 × 10⁹ |
| Xanthomonas campestri | 5.69 × 10⁹ | 7.68 × 10¹⁰ | 4.91 × 10¹⁰ |

### EXAMPLE 5

For each micro-organism a suspension of bio-polymer was prepared as follows: 4 grams of dried xanthan gum was added to 21 grams of water. (Alternatively a 50% suspension of xanthan gum was used.) 25 grams of concentrated biological material (as described for each micro-organism from example 1) was added to this suspension and agitated at room temperature. This was left at room temperature for between 1/2 an hour to an hour.

At the same time 11 grams of acacia gum were added to 14 grams of water and a suspension formed after agitation at room temperature. 25 grams of concentrated biological material was added at room temperature and agitated. This was also kept for 30 to 60 minutes at room temperature after agitation.

The two separate mixtures were added together and kept at room temperature to form a homogenous mix. This solution was kept at room temperature for 1.5 to 2.5 hours, after which a gel was formed. Each gel matrix was stored in plastic bottles at room temperature.

The survival rate of the micro-organisms were tested and the results are set out in the attached Table 5.

TABLE 5
Example 5

| | Initial | Survival | Survival |
|---|---|---|---|
| | Concentration | 1 month | 2 months |
| Organism | cfu g⁻¹ | cfu g⁻¹ | cfu g⁻¹ |
| Serratia entomophila | 5.10 × 10¹⁰ | 2.22 × 10⁷ | 1.17 × 10⁵ |
| Serratia marcescens | 2.38 × 10¹⁰ | 2.76 × 10⁷ | 5.81 × 10⁶ |
| Pseudomonas aeruginosa | 1.01 × 10¹⁰ | 3.71 × 10⁸ | 3.51 × 10⁹ |
| Xanthomonas campestri | 2.83 × 10¹⁰* | 6.86 × 10¹⁰ | 1.51 × 10⁸ |

*theoretical estimate. This is calculated as a function of the weight of the sample, not by a cell assay.

### EXAMPLE 6

The gels of Example 3 were spread to a thickness of 5 mm and left to air dry at room temperature for 15-20 hours. Each dry gel was then stored in the same manner, in a plastic container at room temperature.

The survival rate of the micro-organisms were tested and the results are set out in the attached Table 6.

TABLE 6
Example 6

| | Initial | Survival | Survival |
|---|---|---|---|
| | Concentration | 1 month | 2 months |
| Organism | cfu g⁻¹ | cfu g⁻¹ | cfu g⁻¹ |
| Serratia entomophila | 5.44 × 10¹⁰ | 5.98 × 10⁷ | — |
| Serratia marcescens | 1.31 × 10¹⁰ | 2.42 × 10⁸ | 9.47 × 10⁶ |
| Pseudomonas aeruginosa | 1.09 × 10¹¹ | 4.71 × 10⁹ | 2.58 × 10⁷ |
| Xanthomonas campestri | 2.71 × 10¹⁰ | 1.75 × 10¹⁰ | 6.02 × 10⁹ |

Aspects of the present invention have been described by way of example only and it should be appreciated that modifications and additions may be made thereto without departing from the scope thereof.

The claims defining the invention are:

1. A method for producing a thermo-stable bio-degradable medium for storage of biological materials, said method including the steps of;
(a) preparing at least one bio-polymer at a concentration of 100-10% by weight of a mixture at room temperature, said mixture being in a state selected from a solid and a suspension;
(b) preparing a concentrate of the biological materials of between 10% and 100% (by weight), said concentrate being in a state selected from a solid and a suspension;
(c) combining the mixture of step (a) and the preparation (b), to form a second mix; and
(d) agitating the second mix at room temperature to form a homogeneous suspension; wherein a gel is formed;
and wherein the bio-polymer is selected from the group; xanthan gum; acacia gum; guar gum; gellan; starch; and a combination thereof;
and wherein the biological material is selected from the group: a bio-innoculant, a micro-organism, biological cells, part of a biological cell, parts of a biological cell, a vaccine, at least one pharmaceutical compound, at least one enzyme, at least one hormone, at least one protein; at least one bio-chemical compound, a biologically unstable composition; a chemical composition; and a combination of these.

2. A method of producing a thermo-stable bio-degradable medium for storage of biological materials as claimed in claim 1 wherein the biological material includes: a pesticide; a viricide; a bacteriacide; a fungicide; and a combination of these.

3. A method of producing a thermo-stable bio-degradable medium for storage of biological materials as claimed in claim 1 wherein the biological material is a vaccine selected from: a live vaccine; an oral attenuated vaccine; an encapsulated myco-bacterium vaccine; and a combination of these.

4. A method of producing a thermo-stable bio-degradable medium for storage of biological materials as claimed in claim 3 wherein the vaccine is Bacille Calmette and Guerin (B.C.G.).

5. A method of producing a thermo-stable biodegradable medium as claimed in any one of the preceding claims wherein said biological material is between 10% to 20% by weight in the concentrate of step (b).

6. A method of producing a thermo-stable biodegradable medium as claimed in any one of the previous claims wherein, within step (c) the ratio of the mixtures of steps (a) and (b), which are combined in step (c), is in the range 1:10 to 10:1 by weight.

7. A method of producing a thermo-stable biodegradable medium as claimed in claim 6 wherein the range is 1:1.

8. A method as claimed in any one of the preceding claims wherein the second mix is allowed to stand at room temperature for approximately 60 minutes after step (d).

9. A method of producing a thermo-stable bio-degradable medium as claimed in any one of the preceding claims wherein said method includes, a further step, before step (d); but after step (c) of;
(ci) adding a bio-degradable non-toxic oil to the mix, the concentration of oil being in the range 0.1 to 10% by weight of the mix.

10. A method as claimed in claim 9 wherein the oil is in the range of 1% to 10% by weight of the mix.

11. A method as claimed in claim 9 and 10 wherein the oil used in step (d) is selected from the group of: a monounsaturated oil, a refined oil, a non-refined oil; and a combination of one or more of each of these.

12. A method as claimed in claim 11 wherein the oil used in step (d) is selected from the group: olive oil, canola oil, sunflower seed oil, hydrolysed oils, and a combination thereof.

13. A method of producing a thermo-stable biodegradable medium as claimed in any one of claims 1 to 12, wherein the biological material is selected from cellular organisms and micro-organisms.

14. A method as claimed in claim 13 wherein the cell concentration of the biological material is in the range 10⁵ cells to 10¹² cells g⁻¹.

15. A method as claimed in claim 13 wherein the cell concentration of the biological material is in the range 10⁸ cells to 10¹² cells g⁻¹.

16. A method as claimed in either claim 14 or claim 15 wherein the cell concentration is in the range 10⁹ to 10¹⁰ cells g⁻¹.

17. A method as claimed in claims 1 to 12 wherein the biological material in the concentrate of step (b) is selected from a broth and on a growing medium.

18. A method of producing a thermo-stable bio-degradable medium as claimed in any one of the preceding claims wherein the micro-organism is selected from the group: Serratia, Pseudomonas, Xanthomonas and Rhizobium, and a combination thereof.

19. A method as claimed in any one of the preceding claims wherein the biopolymer is xanthan gum which is added as a dry solid.

20. A method as claimed in any one of claims 1 to 18 wherein the bio-polymer is a mixture of xanthan and acacia gum; which are added as dry solids in a ratio in the range of 1:2 to 1:6 by weight.

21. A method as claimed in any one of the preceding claims where more than one bio-polymer and more than one biological agent is present in the steps (a) to (c).

22. A method of producing a thermo-stable biodegradable medium as claimed in any one of the preceding claims wherein said method further includes; the steps of (a) to (c) with at least one first bio-polymer; the steps of (a) to (c) with at least one second bio-polymer; and a mixing of these two mixtures by steps (c) and (d).

23. A method as claimed in any one of the preceding claims wherein the biological material is selected from a cellular organism and a micro-organism, and wherein storage of the composition is at a stability of better than LT₅₀ with respect to the cell concentration for the length of time of storage.

24. A method as claimed in claim 23 wherein the temperature range of storage is 4° C. to 40° C.

25. A method as claimed in claim 23 wherein the temperature range of storage is between 5° C. to 30° C.

26. A method of producing a thermo-stable bio-degradable medium as claimed in any one of the preceding claims wherein said method includes a further step, (e);
(e) spreading the gel to 5-10 mm in thickness and air-drying it to a moisture content in the range 0.05% to 20% by weight.

27. A method as claimed in claim 26 wherein the time for step (e) is between 12 to 17 hours at ambient temperature.

28. A method as claimed in claim 26 wherein the moisture content is approximately 20% by weight at the end of the drying step.

29. A thermo-stable bio-degradable medium produced by the method as claimed in any one of the preceding claims.

30. A biological storage medium, in the form of a gel of less than 95% by weight of water, produced by method claimed in claims 1 to 25.

31. A liquid spray for application to a substrate (as hereinbefore defined), said spray including;
a portion of thermo-stable biodegradable medium produced by the method as claimed in any one of claims 1 to 27; and
a liquid carrier.

32. A liquid spray for application to a substrate as claimed in claim 31 wherein the medium is added to a trickle irrigation system.

33. A liquid spray as claimed in either claim 31 or 32 for application to a substrate, wherein said substrate is selected from; an agricultural crop; a horticulture crop; a forestry crop; the outer layer of an animal; an uncultivated surface; and a combination thereof.

34. A method of inoculating a plant seed with a biological material, said method including the steps of;
(a) selecting at least one biological material to be used as an inoculant;
(b) preparing at least one medium by the method as claimed in any one of claims 1 to 27;
(c) adding the medium to water and mixing to release the biological material into the solution;
(d) soaking the plant seed in said solution and allowing the biological material to coat the plant seed.

35. A method of inoculating a plant seed with a biological material as claimed in claim 34, said method further including, after step (b), the step;
(bi); adding a powdered compound to the medium, said powdered compound being selected from the group; a second biological material; a dried and powdered bio-polymer containing at least a second biological material; a chemical; and a combination thereof.

36. A method of inoculating a plant seed with a biological material as claimed in either claim 34 or claim 35 wherein the plant seed is dried at room temperature before drilling or seed broadcast.

37. A method of inoculating a seed wherein the seed is drilled in combination with. a dried medium composition as produced by the method of any one of claims 26 to 28.

38. Inoculated seed, inoculated by the method as claimed in any one of claims 33 to 36.
